(19) United States
(12) Patent Application Publication
(10) Pub. No.: US 2026/0330635 A1
(43) Pub. Date: Oct. 22, 2026

(54) SYSTEM AND METHOD FOR SYNCHRONISED POSITIONING OF A CALIBRATION POINTER IN DIFFERENT IMAGES OF THE SAME BREAST

(71) Applicant: HERA-MI, SAINT HERBLAIN (FR)

(72) Inventor: Mickael TARDY, NANTES (FR)

(21) Appl. No.: 19/108,904

(22) PCT Filed: Sep. 8, 2023
(86) PCT No.: PCT/FR2023/051365

§ 371 Date: Mar. 5, 2025

(30) Foreign Application Priority Data
Sep. 9, 2022 (FR) 2209075

(51) Int. Cl. G06T 7/00; A61B 5/055; A61B 6/50; G06V 10/75; G06V 10/764; G06V 10/94

(52) U.S. Cl. CPC G06T 7/0014; A61B 5/055; A61B 6/502; G06V 10/751; G06V 10/764; G06V 10/945; G06T 2200/24; G06T 2207/10088; G06T 2207/10116; G06T 2207/10132; G06T 2207/30068; G06T 2207/30096; G06T 2207/30101; G06V 2201/03

(57) ABSTRACT

A computer-assisted method is provided for synchronously positioning a caibration pointer (Rrx, Rirm) in different acquired images (IMrx, IM_irm) of the same breast (S1). The method includes detecting at least one external structure (SPrx, SPirm) of the breast (S1) in each image, identifying a pair of internal reference structures (M1rx, M1irm) with attached markers, and defining a correlation function according to the pair of internal structures and the external structure in order to match one (Rrx) of the points of the first image (IMrx) with a point (Rirm) of the second image (IMirm). For a pointer positioned on a point (Rrx) of the first image (IMrx), the method uses the correlation function to determine the corresponding point (Rirm) of the second image (IMirm) and displays a pointer on the corresponding point (Rirm) of the second image (IMirm).

### FIELD OF THE INVENTION

The present invention generally relates to synchronizing the positioning of a spatial locating pointer within various images of the same breast.

### PRIOR ART

In order to be able to identify a particular structure in a breast, it is known practice for images to be acquired by means of various methods, for example, using X-ray imaging and using magnetic resonance imaging (MRI), and then for the images of the breast acquired thus to be analyzed.

However, it is complicated and is a potential source of errors for the practitioner to determine which point of an image acquired, for example, using MRI, corresponds to a given point of another image acquired, for example, using X-ray imaging.

A multi-modal position correlation method is known from the prior art that is intended to allow the point of a second acquired image of a breast to be found that corresponds to a given point of a first acquired image of this breast. However, this known position correlation method is mainly based on a general breast deformation model that is used to simulate the deformation of the breast in order to determine the position of the point in the second image.

However, different breasts can exhibit significant differences in terms of deformation behavior, notably according to the internal composition of the breast, such that the use of a breast deformation model as a main method for finding the position of a point in the breast for various acquired images of this breast may not be sufficiently reliable or precise. Furthermore, some acquisition modalities, such as ultrasound imaging, can generate a sequence of images representing the volume of the breast, partly making it difficult to use a general deformation model.

Document EP 2454720 B1 describes a multi-modal breast imaging system and method. The system estimates a volumetric deformation field defining a map between the images based on shape models and a resilient deformation model of the breast.

Document US 2015/363080 A1 describes a system for displaying a plurality of recorded images. A position indication unit allows a user to indicate a position in a first data set displayed in the first display window, in order to obtain a position indicated by the user. A corresponding position determination unit determines a position in a second image data set corresponding to the position indicated by the user, in order to obtain a corresponding position in the second image data set, based on matching information associating positions in the first image data set with matching positions in the second image data set.

The aim of the present invention is to propose a new system and method for overcoming all or some of the problems set forth above.

### SUMMARY OF THE INVENTION

To this end, the aim of the invention is a computer-assisted method for synchronously positioning a locating pointer in various acquired images of the same deformable breast, the method comprising the following steps of:
acquiring a first image, called source image, of the breast according to a first image acquisition configuration;
acquiring a second image, called destination image, of the breast according to a second image acquisition configuration, distinct from the first configuration;
detecting at least one external structure of the breast in the source image and at least one matching external structure, such as the thoracic cage and the skin of the breast, of the breast in the destination image;
detecting and classifying an internal structure of the breast, such as a mass inside the breast, in the source image;
identifying an internal structure in the destination image that corresponds to the internal structure detected in the source image, with said internal structures forming a pair of internal structures;
defining a reference frame attached to said internal structure for each image, with said internal structure to which the reference frame is attached being called reference internal structure;
defining, for several points of the source image, a correlation function for matching one of said points of the source image with a point of the destination image, with the correlation function being established at least as a function of the coordinates of said point of the source image in the reference frame attached to the reference internal structure of the source image, and as a function of the reference frame attached to the reference internal structure of the destination image;
with said correlation function also being established as a function of the distance of said point of the destination image relative to said at least one external structure of the destination image;
displaying, using a graphical user interface, the source image and the destination image;
determining, for a pointer of a pointing device, such as a mouse, positioned by an operator using the graphical user interface on a point of the source image, the corresponding point of the destination image using said correlation function and displaying a pointer on said corresponding point of the destination image.

The method thus allows, by determining an internal structure of the breast as a reference internal structure and at least one external structure of the breast, precise and reliable spatial locating to be provided for a point of the breast for multiple acquisition modalities that allow the content of the breast to be viewed, while dispensing with a compression simulation of the entire breast that would use a complete deformation model of the breast.

The method thus allows precise and reliable synchronization of the positions of the pointers between the various acquired images in real time.

Conversely, in the prior art, transitioning from a position in an image to a corresponding position in another image mainly uses a deformation model of the entire breast, which is a source of inaccuracy.

With the method according to the invention, the position of the point of the pointing device in the destination image is computed in a reference frame attached to the reference internal structure of the destination image that corresponds to a reference internal structure of the source image. The position computation in the reference frame of the reference internal structure allows a precise and reliable computation to be obtained due to the fact that said internal structure acts as a reference anchor relative to said point whose corresponding position is sought after in the destination image. Indeed, the position of the point of the pointing device relative to the reference internal structure in the destination image can be determined more reliably than by using a complex deformation model of the entire breast.

Indeed, the position of the point of the pointing device relative to the internal structure in the destination image of the breast (with said breast in the destination image in practice being deformed relative to the breast of the source image due to its deformable nature), which point corresponds to the point Ri in the source image, can be determined more reliably than by applying a complex deformation model to the entire breast, because the reference internal structure and its local environment are less susceptible to deformation than the entire breast.

In the prior art, the presence of an internal structure is optionally taken into account only in addition to the main application of a general deformation model, but is not used as a main element for determining the position of the pointer, in the sense that in the prior art the position of the pointer of the pointing device in the destination image is not computed in a reference frame attached to the internal structure and by using the position of the pointer of the pointing device in the source image in the reference frame attached to the corresponding internal structure.

The method can also comprise one or more of the following features taken according to any technically permissible combination.

According to one embodiment of the invention, with several pairs of internal structures being identified, the method comprises, for establishing said correlation function, selecting one of the pairs of internal structures, as a pair of reference internal structures, as a function of the type of internal structure of each pair, such as the mass; and, when the pairs of internal structures include several pairs of internal structures of the same type, the pair of reference internal structures is selected by considering, in the source image, the distance of the point relative to the internal structure, preferably an edge of the internal structure, of each pair so as to select the internal structure whose distance between said internal structure, preferably an edge of said internal structure, and said point is the shortest.

According to one embodiment of the invention, the pairs of internal structures that are candidates to be the pair of reference internal structures are, in order of priority, pairs of mass-like structures, calcification cluster-like structures and sets of fibers with architectural disorganization.

Thus, according to one embodiment of the invention, in the event of the detection of a mass pair and a pair of calcification clusters in the source and destination images, the internal structure selected as a reference structure (to which a reference frame is attached in which the position of the point of the pointing device is determined) is the mass.

According to one embodiment of the invention, the first image acquisition configuration and/or the second image acquisition configuration includes a breast deformation parameter, such as a breast compression force applied by an appliance or the gravitational force experienced by the breast, such that the breast in the destination image assumes a shape different from the shape assumed by the breast in the source image; with said correlation function also being established as a function of said one or more breast deformation parameters (resulting from the image acquisition conditions of the destination image and/or of the destination image).

According to one embodiment of the invention, the method comprises determining the type of tissue in which said point of the source image is located, with said correlation function also being established as a function of at least one physical property of said determined tissue type. According to a particular aspect, said physical property of said tissue type is a characteristic physical property of the deformability of the tissue.

In particular, the deformation of the tissue (to which said point belongs) subjected to a force, such as gravity or a compression force applied by an appliance, can be estimated as a function of said deformation parameter resulting from the conditions for acquiring the destination image, and of the type of tissue, since a fatty tissue is more flexible and deformable than a fibro-glandular tissue.

According to one embodiment of the invention, the tissue is of the fatty tissue type, such as fibro-glandular tissue, muscle tissue or calcified tissue.

When the breast of the source image has also experienced a deformation resulting from the conditions for acquiring the destination image, the corresponding deformation parameter also can be considered for establishing the correlation function, preferably in combination with the type of tissue in which the point of the pointing device of the source image is located.

According to one embodiment of the invention, said at least one physical property of said type of tissue in which the point is located and which is used to establish the correlation function is the density or the elasticity.

According to one embodiment of the invention, a mass-like internal structure has a density within a range of predefined values, for example, [1.01; 1.08] g/ml.

The mass also can be identified by its given closed geometric shape, for example, round or oval, with the shape allowing various sub-types of masses to be distinguished, such as a ganglion relative to a malignant mass.

Generally, the internal structures can differ from each other in terms of their density, optionally in combination with their geometric shape.

According to one embodiment of the invention, said external structure used to establish the correlation function comprises the skin of the breast that defines the contour of the breast.

According to one embodiment of the invention, the first image acquisition configuration and the second image acquisition configuration differ from each other by at least one of the following parameters:
the acquisition technology used;
the acquisition parameters or conditions used;
the acquisition time.

According to one embodiment of the invention, the first image acquisition configuration and the second image acquisition configuration comprise at least one of the following acquisition methods:
X-ray imaging;
magnetic resonance imaging;
ultrasound imaging.

According to one embodiment of the invention, the first image acquisition configuration and the second image acquisition configuration comprise, on the one hand, an X-ray imaging or magnetic resonance imaging acquisition method, and, on the other hand, an ultrasound imaging acquisition method.

The definition or selection of a reference internal structure as provided in the method according to the invention for establishing the correlation function in order to determine, in the destination image, the point of the breast that corresponds to a point of the breast of the source image, is particularly effective in the case of a source image acquired using MRI imaging or using X-ray imaging and a destination image acquired using ultrasound imaging, as opposed to the solutions of general breast deformation models that are difficult to apply for determining the matches between elements of the breast in these images, since acquisition using ultrasound imaging generally results in partial imaging of the breast.

The invention also relates to a non-transitory computer program product comprising program code instructions for executing the steps of a method according to any one of the preceding embodiments, when said program is executed by a processor of a computer system.

The invention also relates to a computer system comprising a memory containing program code instructions for executing the steps of a method according to any one of the preceding embodiments, and a processing unit comprising a processor for executing said program code instructions, said computer system further comprising:
a memory for storing image data;
a pointing device, such as a mouse, that can be handled by an operator;
a display screen; and
a graphical user interface configured to:
simultaneously and separately display a first acquired image and a second acquired image of the same breast, with the first image forming the source image and the second image forming the destination image;
display, on a point of the first image, a pointer as a function of the position of the pointing device handled by the operator;
display, following the execution of said program code instructions, another pointer on the acquired second image at a point of said second image that corresponds to the point of the first image on which the pointer of the pointing device handled by the operator is positioned.

The invention also relates to a facility comprising:
a computer system as proposed above;
a first imaging appliance for acquiring the first image; and
a second imaging appliance, different from the first imaging appliance, for acquiring the second image.

According to one embodiment, said first imaging appliance comprises an X-ray appliance that comprises an emitter device able to emit an X-ray beam and a receiver device able to receive the X-rays emitted by the emitter device after they pass through the breast, with the X-ray appliance being configured to transmit image data to the computer system that corresponds to the X-rays received by the receiver device after they pass through the breast.

According to one embodiment, said second imaging appliance comprises a magnetic resonance appliance that comprises an emitter device able to emit magnetic radiation and a receiver device able to receive the magnetic radiation emitted by the emitter device after it passes through the breast, with the magnetic resonance appliance being configured to transmit image data to the computer system that corresponds to the magnetic radiation received by the receiver device after it passes through the breast.

### BRIEF DESCRIPTION OF THE DRAWINGS

Further features and advantages of the invention will become more clearly apparent from the following description, which is purely illustrative and non-limiting and should be read with reference to the appended drawings, in which:

FIG. 1 is a schematic view of an X-ray imaging appliance;

FIG. 2 is a schematic view of a magnetic resonance imaging appliance;

FIG. 3 is a schematic view of an ultrasound imaging appliance;

FIG. 4 is a series of schematic views of an acquired image of a breast, with various computer processing operations being applied to said image so as to detect a vein-like internal structure;

FIG. 5 is a series of schematic views of an acquired image of a breast, with various computer processing operations being applied to said image, including filtering of fine and extended structures and smooth rendering filtering, so as to detect a mass-like internal structure;

FIG. 6 is a series of schematic views of an acquired image of a breast, with various computer processing operations being applied to said image, including shape recognition, so as to detect a mass-like internal structure;

FIG. 7 is a schematic view of an acquired image of a breast, with shape recognition processing being applied on certain parts of the image in order to recognize various types of internal structures;

FIG. 8 is a schematic view of two acquired images of a breast acquired using different acquisition modalities, with matching of any internal structures (such as a mass, a calcification cluster) identified in the two images and matching of any external structures (the skin and the thoracic cage) identified in the two images;

FIG. 9 is a schematic view of two acquired images of a breast acquired using different acquisition modalities, namely, X-ray acquisition and MRI acquisition with a sagittal projection view, involving the generation of a reference or coordinate system, called general system, attached to the breast, with the reference frames in the two images being matched;

FIG. 9A is a schematic view of two acquired images of a breast acquired using different acquisition modalities, namely, X-ray acquisition and MRI acquisition with a transverse projection view, involving the generation of a reference or coordinate system, called general system, attached to the breast, with the reference frames in the two images being matched;

FIG. 10 is a schematic view of two acquired images of a breast acquired using different acquisition modalities, illustrating a correlation function T established between the structures identified in the two images;

FIG. 11 is a schematic view of two acquired images of a breast acquired using different acquisition modalities, involving, for each image, the generation of a reference or coordinate system attached to an internal structure of the breast identified in the image, with said internal structure being paired with an internal structure identified in the other image, and involving establishing a correlation function between points of the two images as a function of the reference systems attached to the identified internal structures of the breast;

FIG. 11A is a detailed view of a pair of associated internal structures identified in the images of FIG. 11, with their associated reference frames, and the synchronization of the position of a locating pointer for the two internal structures using the position of the pointer in the reference frame of the internal structure;

FIG. 12 is a schematic view of two acquired images of a breast acquired using different acquisition modalities, with a first locating pointer being positioned by the operator between a mass (internal structure) and the skin (external structure) of the breast of the first image (source image), and a second locating pointer being correspondingly positioned between the mass and the skin of the breast of the second image (destination image) in a synchronized manner with the first pointer;

FIG. 13 is a schematic view of two acquired images of a breast acquired using different acquisition modalities, with a first locating pointer being positioned by the operator between a mass (internal structure) and a calcification cluster (other internal structure) of the breast of the first image (source image), and a second locating pointer being correspondingly positioned between the mass and the corresponding calcification cluster of the breast of the second image (destination image) in a synchronized manner with the first pointer;

FIG. 13A is a detailed view illustrating the distance between the first locating pointer and the mass and the distance between the first locating pointer and the calcification cluster of the first image (source image) of FIG. 13, with an additional mass also being added to illustrate the fact that, for two internal structures of the same type identified in the image, the internal structure that is selected to act as the reference internal structure is determined as a function of the distance between the point of the pointer and the internal structure;

FIG. 14 is a schematic view showing a facility according to one embodiment of the invention comprising a computer processing system and imaging appliances configured to acquire and provide the computer processing system with various images of a breast.

### DETAILED DESCRIPTION

The invention is described in greater detail hereafter with reference to the accompanying drawings, which illustrate embodiments of the invention. In the drawings, the size and the relative sizes of the elements can be exaggerated for the sake of clarity. Similar numbers refer to similar elements throughout all the drawings. However, the invention can be implemented in many different forms and should not be understood to be limited to the embodiments disclosed herein.

A reference throughout the specification to “an embodiment” means that a functionality, a structure, or a particular feature described in relation to an embodiment is included in at least one embodiment of the present invention. Thus, the occurrence of the expression “in one embodiment” at various points throughout the specification does not necessarily refer to the same embodiment. Furthermore, the functionalities, the structures, or the particular features can be combined in any suitable manner in one or more embodiments.

With reference to the figures, a method and a computer system are proposed for synchronously positioning locating pointers (also called cursors) within a plurality of acquired images of a breast. The images are computer images.

Spatial locating within a plurality of images of the same physical breast allows, on a graphical user interface, the location of a locating pointer to be synchronized, with said locating pointer being able to move by means of a pointing device, such as a mouse, with the location of another locating pointer on a corresponding point of another image. The remainder of the description is set forth for the case of two images of the same breast acquired using different modalities, but can be applied to a larger number of images acquired using different modalities, as well as to two or more images of the same breast acquired according to the same modality and different timeframes.

### Deformable Object

The studied deformable object is a breast. The breast is a resilient and deformable object, which can react to the natural and artificial external forces applied thereto, such as gravity or a compression force. The breast can be characterized by its physical properties, such as its dimensions, its elasticity, its density, its mass.

The breast can be studied using different imaging techniques, also called imaging modalities. The acquisitions generated by these modalities can be carried out under different conditions or settings. The same acquisition modality can be used using different settings or at different times resulting in different acquisition conditions.

The conditions can include different time moments, different spatial positions, different states of the breast (for example, related to temperature variations between acquisitions), etc. An object that is characterized by its own physical properties can behave differently depending on the acquisition conditions. Furthermore, each type of imaging modality, i.e., each image acquisition technique or method, is based on different physical principles resulting in different behaviors of the breast.

Thus, for a studied breast, the set of imaging appliances generates a series of acquisitions that can show the breast in different aspects. It is thus possible to view structures of the same type differently, for example, breast tissues.

The described method allows a link (match) to be established between the acquired images in order to browse the images, in a synchronized manner, such that when a pointer is located on a point of a first image, a corresponding pointer is displayed on a point (obtained precisely and reliably, as explained hereafter) of the other or of each other image that corresponds to the selected point of said first image (i.e., the point on which the pointer of the operator-movable pointing device is located).

### Images

An acquired image can be a 2D or 3D image. In the case of a 3D image, the image displayed on the screen is a projection of the image in a given plane. The image is acquired with an imaging appliance that allows an image of a studied breast to be acquired and viewed that has been subjected to emitted waves or radiation, and optionally to a force (such as a compression force) that deforms the breast. The appliance allows the external and internal aspects (structures) of the studied breast to be viewed.

An image is defined on the computer plane as a set of points in space. Said set is characterized by dimensions W×H×D, where W is the width, H is the height, and D is the depth of the image.

A value V is assigned to each point of the image, which value can be a scalar (i.e., a single numerical value), vector or matrix (group of numerical values) value.

The numerical values can belong, for example, to a subset of the space of natural N or real R numbers.

An image can be associated with a set of acquisition parameters, such as parameters of exerted forces or radiation emitted on the breast, or the physical properties of the studied breast measured at the time of acquisition.

### Image Acquisition Method

The methods for acquiring an image can include all or some of the following methods.

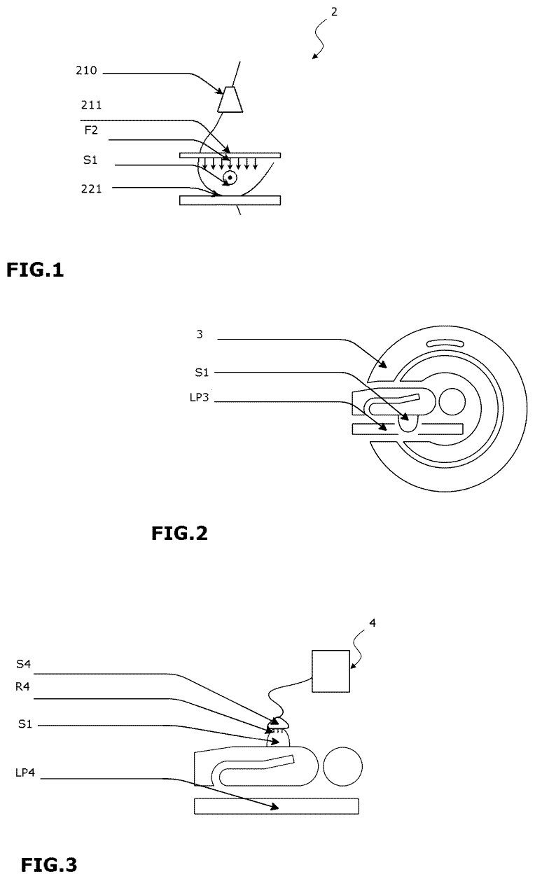

As illustrated in FIG. 1, a breast image can be acquired by an X-ray imaging method using an X-ray imaging appliance 2.

The X-ray imaging appliance comprises a breast compression system that includes a compression plate 211 and a support plate 221, between which a breast S1 is compressed with a force F2, an X-ray emission system 210 for emitting X-rays through the breast, and a reception system 220 for receiving the X-rays that have passed through the breast. The acquisition can be carried out over varying angles of incidence relative to the axis of the studied breast.

As illustrated in FIG. 2, an image of the breast S1 of a person lying on their stomach on a bed LP3 can be acquired using a Magnetic Resonance Imaging (MRI) imaging method using a magnetic resonance imaging appliance 3.

As illustrated in FIG. 3, an image of a breast of a person lying on their back on a bed LP4 can be acquired using a 2D or 3D ultrasound imaging method (i.e., ultrasound imaging), using an ultrasound imaging appliance 4 that comprises a probe S4 configured to emit ultrasound R4 in the breast S1.

Depending on the spatial position, the studied breast can experience external forces, such as gravity, resulting in deformation, such as stretching or compression.

Preferably, for each implemented image acquisition configuration, the acquisition parameters used to implement said acquisition (for example, compression force, radiation intensity, contrast agent used, etc.) are stored in a memory of a computer system.

The images acquired by the various appliances 2, 3 are transmitted to a computer system 1 (illustrated in FIG. 14).

As illustrated in FIG. 14, the computer system 1 comprises a memory 101 containing program code instructions for executing the steps of a method described hereafter, and a processing unit 102 for executing said program code instructions.

Said computer system also comprises a memory 103 for storing image data, a pointing device 105, such as a mouse, that can be handled by an operator, and a display screen 104. According to one embodiment, the memories 101 and 103 can be fully or partly shared or can be separate.

The computer system 1 also comprises a graphical user interface allowing, as described hereafter, first and second acquired images of the same breast to be displayed simultaneously and separately, and allowing a pointer to be displayed on the first image as a function of the position of the pointing device handled by the operator, while displaying another pointer on the second acquired image at a point of said second image that corresponds to the point of the first image on which the pointer of the pointing device handled by the operator is positioned.

The computer storage memory for storing the acquired images also preferably allows the corresponding acquisition parameters to be stored.

Thus, the one or more imaging appliances allow a series of acquisitions to be generated showing the breast according to various aspects, and thereby allows the same structures, such as structures of the tissue, vessel, mass type, etc., to be viewed differently.

The memory used for saving the acquired images allows the set of acquired images to be written, irrespective of the number of dimensions of the image, such as 2D, 3D, or 4D, and allows the set of acquisition parameters associated with the completed image acquisitions to be stored.

Reading an image from the memory allows the image to be read with its acquisition parameters.

Other image acquisition methods also can be used, such as computed tomography or positron emission tomography (PET) image acquisition.

### Advantage of Acquisitions Using Different Methods

Acquiring images of the breast using different methods allows various images and thus different renderings of the breast to be obtained, thereby highlighting particular aspects (structures) of the studied breast.

For example, X-ray imaging allows the response of the breast tissues to the emission of the X-rays to be illustrated, reflecting the density of the tissues.

Ultrasound imaging allows the response to ultrasound emitted by a probe to be illustrated, thereby reflecting the state of the tissues, such as a liquid, soft, solid state.

### The Breast

The structures of a breast that can be worthwhile detecting and classifying can include all or some of the following structures:
the skin;
the nipple;
the pectoral muscle;
the sub-mammary furrow;
the masses; and/or
micro-calcifications and macro-calcifications.

More generally, the structures to be classified include:
one or more anatomical structures (for example, nipple, skin);
one or more physiological structures (for example, vessels); and/or
one or more pathological structures (for example, masses).

### Method for Synchronously Positioning Locating Pointers

The method for synchronously positioning locating pointers on various images is described hereafter for two images, but obviously applies to a larger number of images.

The method can comprise the following steps described with reference to an example illustrated in FIG. 11. It is understood that the steps are applicable to the other examples.

A first image IM_irm of the breast S1 (source image) is acquired according to a first image acquisition configuration. The first configuration comprises a given acquisition method, for example, using MRI, with a given setting.

The image IM_irm illustrated in FIG. 11 is the sagittal projection view of the MRI image of the breast that is acquired in 3D.

A second image IM_rx of the breast S1 (destination image) is acquired according to a second image acquisition configuration, distinct from the first configuration.

According to one embodiment, the second configuration comprises another acquisition method, for example, using X-ray. As a variant, the second acquisition configuration can comprise the same first acquisition method but with a different setting or acquisition conditions (time moment, spatial position, temperature, deformation).

The image IM_rx illustrated in FIG. 11 is an image of the breast acquired using X-ray.

The images are stored in a computer memory.

### Segmentation and classification

According to one embodiment, one or more processing operations is/are applied to each acquired image to allow various structures of the breast that can be identified on the images to be segmented (detected) and classified (determine the type).

The structures can be of the following type:
anatomical structure, for example, the nipple, the skin;
physiological structure, for example, a vessel;
pathological structure, for example, a mass.

The segmentation and classification processing operations are applied to each image in order to be able to:
isolate the points relating to an identifiable structure;
associate a category (type) with each of the identified structures.

The applied computer processing operations allow the breast or an element of interest on the image to be segmented (delineated), which allows one or more external structures to be detected in order to determine the limits (contour) of the breast. As explained hereafter, considering one or more external structures of the breast allows a constraint to be applied for determining the position of the pointer and for checking that a pointer positioned inside the breast for an image acquired according to a given configuration is not positioned outside the breast for another image acquired according to another configuration. An external structure, for example, the skin of the breast, forms at least one portion of the contour of the breast.

The computer processing operations also allow regions inside the studied breast to be segmented in order to classify (assign a category or type, i.e., identify) the regions.

A variable number of processing operations can be applied to an image in order to assign one or more features to each point of the image that relate to the type of structure associated with said point.

The processing operations applied to the image can include filtering applications, such as, for example, noise attenuation or contrast enhancement.

Each processing operation can be applied to all or some of the image. The acquisition parameters also can be used to define the processing to be applied.

According to the objectives, for example, according to the type of structures to be identified, different series of processing operations can be implemented.

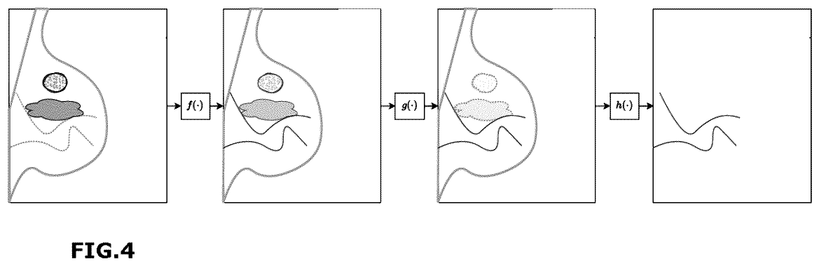

For detecting a vessel structure, and as illustrated in FIG. 4, the next series of processing operations can be applied to the entire image:
applying a mathematical function f(⋅) to the intensity values of the set of points of the image for highlighting extended and fine structures, for example, by enhancing intensities of points associated with said extended and fine structures;
applying another mathematical function g(⋅) to the intensity values of the set of points of the image allowing masking of smooth and bulky structures, for example, by lowering intensities of points associated with said smooth and bulky structures;
applying a thresholding function h(⋅) to the intensity values allowing isolation of the extended and fine structures, in order to reveal vessel-like structures.

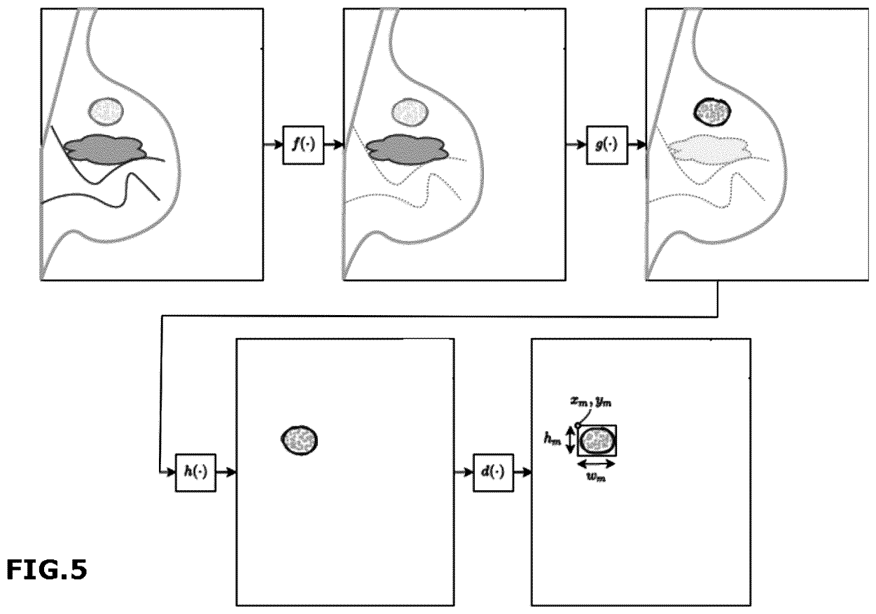

Similarly, pathological structures such as masses can be highlighted, as illustrated in FIG. 5, with a series of processing operations comprising a function f(⋅) corresponding to filtering fine and extended structures, a function g(⋅) corresponding to filtering smooth-rendered structures, a function h(⋅) corresponding to an intensity thresholding function and a function d(⋅) corresponding to a function for extracting coordinates and object size from a thresholded image. The result of the series of processing operations allows the points associated with the searched structure to be identified and consequently allows the coordinates (xm, ym) and the dimensions (hm, wm) of said structure to be identified.

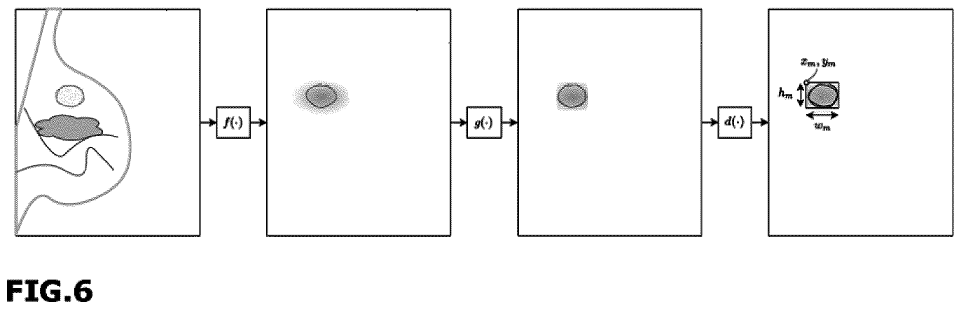

Alternatively, the processing operations can be based on shape recognition functions for detecting structures. Such a series of processing operations is illustrated in FIG. 6 with a series of processing operations comprising a function f(⋅) corresponding to a shape recognition function, a function g(⋅) corresponding to an intensity thresholding function on the result of the function f(⋅) and a function d(⋅) corresponding to a function of extracting coordinates and object size from the thresholded result of the shape recognition function. Similarly, the result of the series of processing operations allows the points associated with the searched structure to be identified and consequently allows the coordinates (xm, ym) and the dimensions (hm, wm) of said structure to be identified.

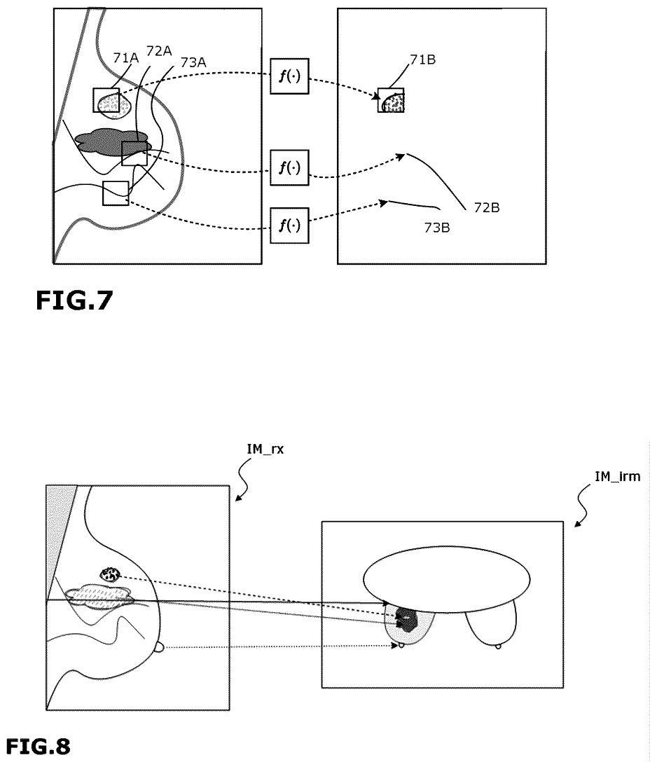

As illustrated in FIG. 7, the recognition function f(⋅) for the shapes 71B, 72B, 73B can be based on a combination of mathematical operations applied to corresponding portions 71A, 72A, 73A of the image. The purpose of said mathematical operations is to detect the shapes characterizing the searched structures, such as masses.

Thus, as illustrated in FIG. 7, locating a known shape can result in the enhancement of the intensity values of the points associated with said shape, while in the case of a non-identified shape the intensity values will be lowered.

The type of structure can be defined by various means that may or may not be combined. By way of an example, these means can comprise one or more image processing operations as explained above, comparing a property of the structure, such as its density and/or its shape, with reference data of structures stored in a database and accessible by a computer system.

The density of a structure can notably result in intensity values of different pixels in the image. Depending on the type of imaging, denser structures can have lower or higher intensity values.

### Image Association and Matching Structures

The various acquired images of the breast allow the studied breast to be viewed from various aspects. However, a certain number of properties are shared between the images generated by the various image acquisition configurations of the breast.

Indeed, with respect to the same breast, the image of which has been acquired using various configurations, at least one portion of the structures of the breast can be viewed on the various images.

Analyses of pairs of structures present in two different acquisition images are carried out in order to match the identified structures.

According to one embodiment, an association is made between the various types of acquired images.

A first association thus can be defined between a first image (source image) of the breast acquired using X-ray imaging and a second image (destination image) of said breast acquired using MRI imaging.

A second association can be made between a first image (source image) of the breast acquired using X-ray imaging and a second image (destination image) of the breast acquired using ultrasound imaging.

A third association can be made between a first image (source image) of the breast acquired using MRI imaging and a second image (destination image) of the breast acquired using ultrasound imaging.

For each association of images, each image is analyzed in order to select the structures that are present on the two images and that can be matched. In the case of the breast, provision can be made for all or some of the following structures to be matched:
the skin;
the nipple;
the pectoral muscle;
the sub-mammary furrow;
the masses;
the calcification clusters, the micro-and macro-calcifications;
the vessels.

Preferably, a sorting step is carried out to eliminate the least probable matches between several candidates. The sorting can be based on the properties of the images (for example, visual aspects), as well as on the acquisition parameters, notably the spatial features of the breast (related to the position of the individual).

The result of the analysis carried out for a radiological image and a magnetic resonance image is illustrated in FIG. 8. Each arrow connects a structure identified in an image that corresponds to a structure identified in the other image.

Identified structures, such as the thoracic cage, the nipple, a mass, or a calcification cluster thus can be matched in various images.

Thus, following the step of matching identifiable inter-acquisition structures, unique pairs of structures are identified. As explained hereafter, in order to synchronize the position of a pointer between the source image (on which the user positions a locating pointer using a pointing device such as a mouse), and the destination image (for which the corresponding position of the pointer is to be determined), the one or more pairs of internal structures is defined or selected as a pair of reference internal structures for defining a reference frame (coordinate system) attached to the corresponding internal structure in order to reliably determine the position of the pointer in the destination image based on the position of the pointer in the source image.

The paired structures comprise paired internal structures and paired external structures.

As noted above, the external structures can notably include:
the nipple;
the skin;
the thoracic cage/pectoral muscle;
the sub-mammary furrow;
the axillary cavity.

The internal structures can notably include:
a mass;
a calcification cluster (the calcifications can be micro-calcifications).

### Coordinate System Attached to an External Structure

According to one embodiment, among the paired external structures, a pair of external structures can be retained in order to define a coordinate system (general reference frame) in the corresponding images.

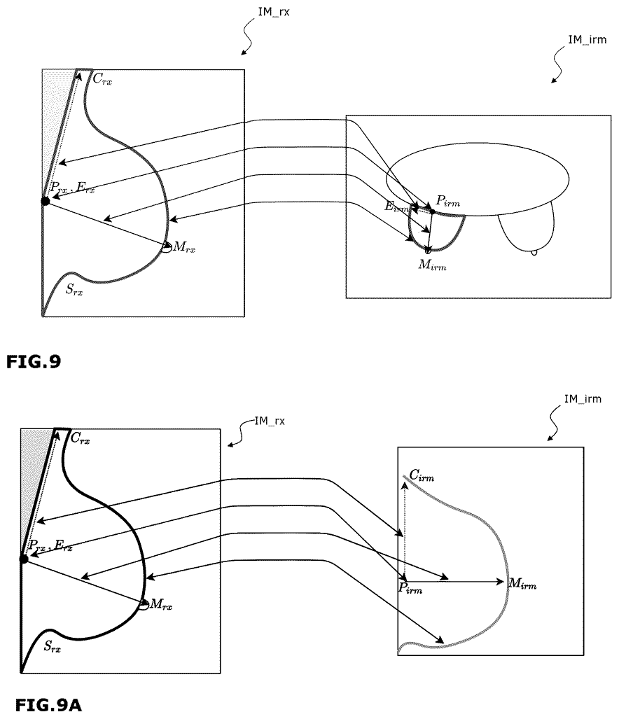

The result of defining a coordinate system applied to an external structure for a breast for imaging modalities, such as radiological imaging and magnetic resonance imaging, is illustrated in FIG. 9 and FIG. 9A.

By way of an example with reference to FIG. 9, for a breast, the origin Prx; Pirm of a coordinate system associated with an external structure can be the midpoint of the thoracic cage relative to the breast.

Other paired external structures can be employed to define the orthogonal orientation axes Crx, Mrx, Erx; Mirm, Eirm of the corresponding reference frame. For a breast, the axes can be defined between the origin and the nipple, and between the origin and the axillary cavity.

One or more external structures can be retained to act as limits of the breast. The limits of the breast can be defined by the skin surrounding the breast, the nipple and/or the thoracic cage.

The arrows that connect the two images indicate the matches between:
the origin Prx and the origin Pirm;
the major axis Mrx and the major axis Mirm;
the limit corresponding to the contour of the breast in each image;
the minor axis Erx and the minor axis Eirm.

For three-dimensional imaging, such as MRI, the coordinate system for said imaging is defined in three dimensions.

As explained hereafter, the reference frame attached to the reference internal structure can be defined as a function of the general reference frame attached to an external structure (such as the thoracic cage) of the breast.

### Internal Structure Detection

The method comprises, for each acquired image, a step of detecting an internal structure.

An internal structure is understood to mean an element contained in the envelope (or contour) of the breast. Therefore, the internal structure is distinct from the envelope of the breast that forms an external structure. The envelope of the breast comprises the external portion formed by the skin of the breast that is able to be in contact with an external element used to deform said breast. A portion of the external structure of the breast also can be considered to be formed by a portion of the thoracic cage that defines a portion of the envelope of the breast.

An internal or external structure can be detected by a processing operation or by a series of processing operations for the image, as explained above.

After detecting an internal structure, a classification step is carried out in order to define the internal structure. The internal structure can be defined by assigning a class (type) to each detected internal structure.

For a breast, the classes that can be assigned to an internal structure can include all or some of the following classes:
mass (malignant mass, ganglion);
calcification cluster, micro-calcification; macro-calcification;
set of fibers with architectural (structural) disorganization.

The viewed structures can be classified by considering acquisition parameters. The classification step can be carried out by applying a series of image processing operations to each acquired image and/or by comparing (correlating) with reference internal structure data stored in a database.

Once the internal structures have been classified, at least one pair of internal structures StInt1irm; StInt1rx is identified in the two images (illustrated in FIG. 11).

As for the external structures, a pair of internal structures can be identified by correlation by identifying an internal structure in an image that corresponds to an internal structure in another image. To this end, the system comprises a correlation computer module.

In the event that one image contains several candidates for the structure that are likely to form the pair of internal structures, an operation involving sorting and rejecting candidates can be carried out according to acceptability or probability criteria.

Thus, as is schematically shown in the example of FIG. 11, each pair of identified internal structures comprises an internal structure StInt1irm in the first image IM_irm (source image) that corresponds to an internal structure StIntrx of the second image IM_rx (destination image).

Advantageously, corresponding internal structures can share all or some of the following data:
the type (class) of the structure;
the size;
the position relative to the envelope of the breast (external structure).

### Coordinate System Attached to an Internal Structure

A coordinate system (reference frame) is attached to an internal structure for each image in order to act as a reference internal structure. When several pairs of internal structures are identified, an operation involving selecting a reference internal structure is carried out, as described hereafter. The coordinate system comprises an origin and orientation axes.

In the example that is illustrated in FIG. 11 and is applicable to the other examples, for the magnetic resonance image (MRI) as a sagittal projection (left-hand image in FIG. 11), an origin Omirm is associated with the mass-like internal structure StInt1irm, on which the pointer of the pointing device handled by the operator is positioned at a point Rirm.

The origin of the reference frame associated with the internal structure StInt1irm can be the barycenter or the geometric center of the internal structure.

The axes Xmirm and Ymirm are axes of the coordinate system that can be established as a function of the axes of a general spatial reference frame (i.e., associated with the individual) known and stored when acquiring the image (or established as a function of the characteristic transverse axes, for example, the longitudinal axis, of the internal structure, or can be established as a function of the axes of the reference frame associated with an external structure, in this case, Pirm, Cirm, Mirm, in particular an external structure of the breast such as the thoracic cage that defines a portion of the breast, the skin and/or the nipple).

The reference frame also can be defined as a function of the type of internal structure. Thus, for a vessel-like internal structure, provision can be made for the reference frame to comprise a main axis defined by the longitudinal axis of the vessel.

For the mediolateral oblique view X-ray image of FIG. 11 (right-hand image), an origin Omrx is associated with the mass-like internal structure that corresponds to the internal structure StInt1rx.

The axes Xmrx and Ymrx are axes of the coordinate system that can be established as a function of the axes of a reference frame associated with the known individual and stored when acquiring the image (or established as a function of the characteristic transverse axes, for example, the longitudinal axis, of the internal structure, or established as a function of the axes of the reference frame associated with the external structure, in this case, Prx, Crx, Mrx).

The left-hand and right-hand images of FIG. 11A are detailed views of the internal structure StInt1irm identified in the left-hand image of FIG. 11 with the positioning point Rirm of the pointer of the pointing device, in the reference frame Omirm, Xmirm and Ymirm associated with the internal structure StInt1imm, and respectively of the corresponding internal structure StInt1rx identified in the right-hand image of FIG. 11 with the corresponding point Rrx of the pointer in the reference frame Omirm, Xmirm and Ymirm.

### Correlation Function

For each acquired image association (i, j), a correlation function Tij (or matching function) is defined in order to match each point of an acquired image i, called source acquisition i, with a point of an acquired image j, called destination acquisition j. In other words, said correlation function is a function whereby, for a given position of the pointer of the pointing device in the breast of a source image, the corresponding position of the pointer to be displayed in the breast of the destination image is obtained.

The correlation function Tij is defined as a function of at least one pair of internal structures identified in the source and destination images, and in particular as a function of a pair of internal structures selected as a pair of reference internal structures. A reference frame is attached to each internal structure such that, for the correlation function, the position of the pointer inside the breast of the source image is considered in the reference frame attached to the reference internal structure of the source image, and the position of the pointer in the destination image is defined in the reference frame attached to the reference internal structure of the destination image.

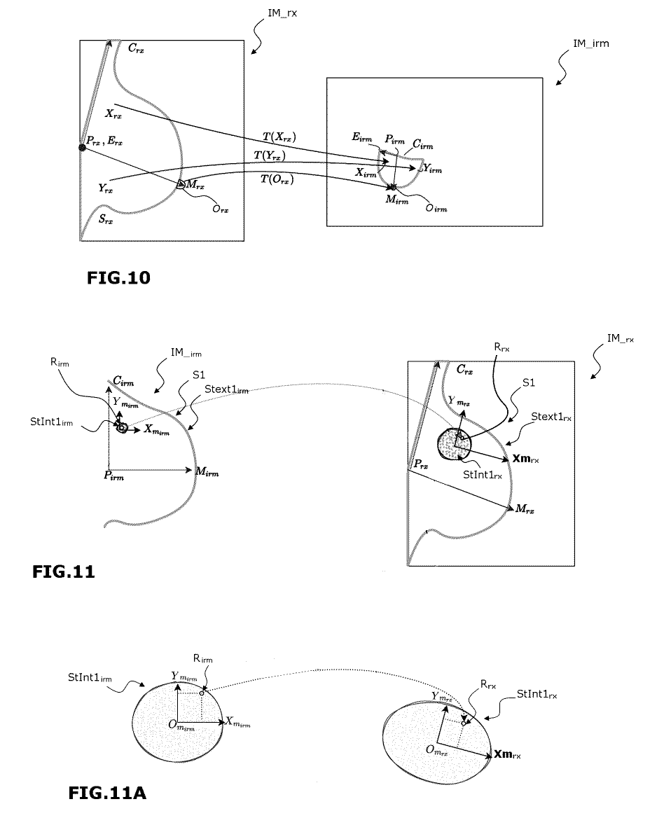

The result of applying a correlation function T(Xrx); T(Yrx), T(Orx) for points Xrx; Yrx, Orx of external structures of a breast, for the left-hand image of FIG. 10 (source image obtained by magnetic resonance imaging (MRI)), is illustrated in this FIG. 10 with the obtained points Xirm; Yirm, Oirm in the right-hand image (destination image obtained using X-ray imaging).

The correlation function Tij also can be established as a function of additional data including all or some of the following data:
internal or external structure data used to define a reference frame;
one or more other structures that have been analyzed;
one or more acquisition parameters, as well as one or more physical properties of the imaging modalities.

### Consideration of the Internal Structure

Considering a pair of reference internal structures identified on the source acquisition and the destination acquisition, for the spatial correlation of the pointer, enables more precise and reliable positioning of the pointer in a second acquired image of the breast (the destination image) matching the position of the pointer of the pointing device handled by the operator in a first acquired image of the breast (the destination image), as explained hereafter.

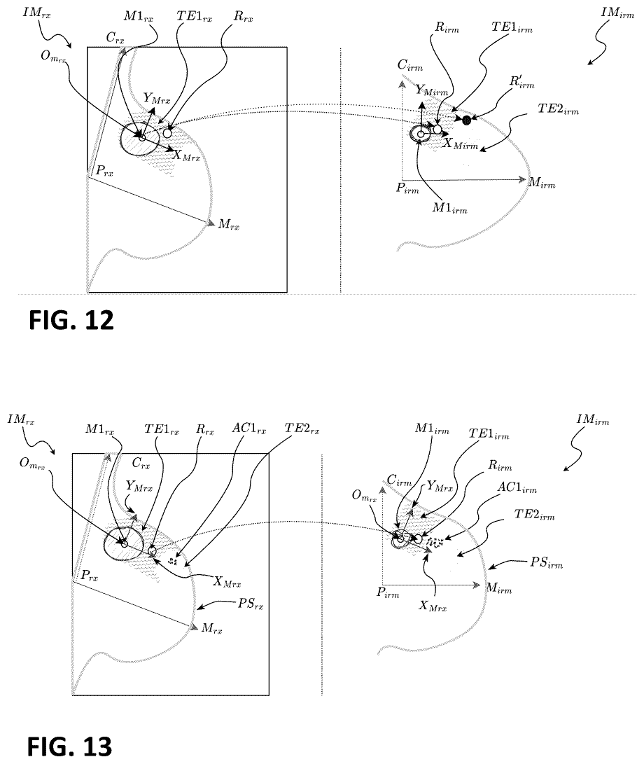

An example is described hereafter with reference to FIG. 12 that highlights the benefit of considering a pair of reference internal structures for precisely and reliably determining the coordinates (position) of the cursor (locating pointer) in the destination image that correspond to the coordinates (position) of the cursor (locating pointer) in the base image.

The left-hand image, denoted IMrx, of FIG. 12 corresponds to an image of a breast acquired using 2D X-ray imaging (mammography), and the right-hand image, denoted IMirm, corresponds to an image of the breast acquired using MRI as a sagittal projection.

As illustrated in the examples and as is applicable to any embodiment of the invention, coordinate systems (reference frames) are defined in said image acquisitions along the PM (defined by the points P and M), PC (defined by the points P and C) and optionally PE (defined by the points P and E, with E, not shown, being defined as a point on the minor axis that is orthogonal to the axes PM and PC) axes by being associated with one or more external structures of the breast, such as the thoracic cage, the skin and/or the nipple.

Irrespective of the embodiment of the invention, it is possible to define the axis PM as passing through the nipple and having to be perpendicular to the pectoral plane to which the axis PC belongs.

In the example illustrated in FIG. 12, the dimensions of the views according to the acquisitions are:
PM=10 cm; PC=5 cm; for MRI;
PM=8 cm ; PC=6 cm; for mammography.

The image IMirm acquired using MRI is a destination acquisition, and the image IMrx acquired using mammography is a source acquisition.

An internal structure M1rx is identified in the image IMrx. As previously explained, the internal structure M1rx, in this case a mass, can be identified by image processing and/or by comparing with reference data stored in a database. A reference frame is attached to this internal structure M1rx, with the reference frame comprising an internal structure center O_Mrx and the associated coordinate system X_Mrx and Y_Mrx. The center O_Mrx is set to O_Mrx_x=18 mm along the axis PM and to O_Mrx_y=28 mm along the axis PC.

As illustrated in the examples and as is applicable to any embodiment of the invention, the center O can be defined as the barycenter or the geometric center of the mass, and the associated axes can be defined as being parallel to the axes PM and PC attached to an external structure of the breast, such as the thoracic cage and/or the skin.

An internal structure M1irm is identified in the image IMirm, such that the internal structure M1rx corresponds to the internal structure M1irm. A reference frame is attached to this internal structure M1irm, the reference frame comprising the center OMirm of said internal structure and the associated coordinate system XMirm, YMirm are defined such that the center OMirm is set to OMirm_x=10 mm along the axis PM and OMirm_y=25 mm along the axis PC.

A reference point Rrx (positioning point of the pointer) is placed by the operator, for example, using a mouse, in the source image IMrx. By way of an example, the reference point Rrx has the following coordinates R_S1_rx_x=25 mm along the axis PM and R_S1_rx_y=30 mm along the axis PC (reference frame of the breast S1).

Said reference point Rrx of the pointer of the pointing device is also identified in the coordinate system of the internal structure M1rx. By way of an example, the reference point Rrx has the following coordinates R_Mrx_x=10 mm along the axis XMrx and R_Mrx_y=5 mm along the axis YMrx of the reference internal structure M1rx.

A correlation function (mathematical operation) is defined to allow a point of the image IMrx to be positioned toward a corresponding point of the image IMirm (in other words, to determine the position of a point in the destination image IMirm that corresponds to a given point in the source image IMrx). The correlation function considers the pair of internal structures M1rx, M1irm and the reference frames attached to these internal structures as reference internal structures. The correlation function particularly considers the position of the point of the image IMrx in the reference frame attached to the reference internal structure M1rx of the source image IMrx. The correlation function also considers the distance between the point Rirm and an external structure PSirm, such as the skin (and/or the nipple and/or the thoracic cage), for example, in order to check that the determined point Rirm is indeed inside the breast and not outside. It is also possible for the correlation function to consider the distance between the point Rrx and an external structure in the source image IMrx in order to be able, for example, to compare this distance with that of the determined point Rirm relative to the matching external structure in the destination image IMirm.

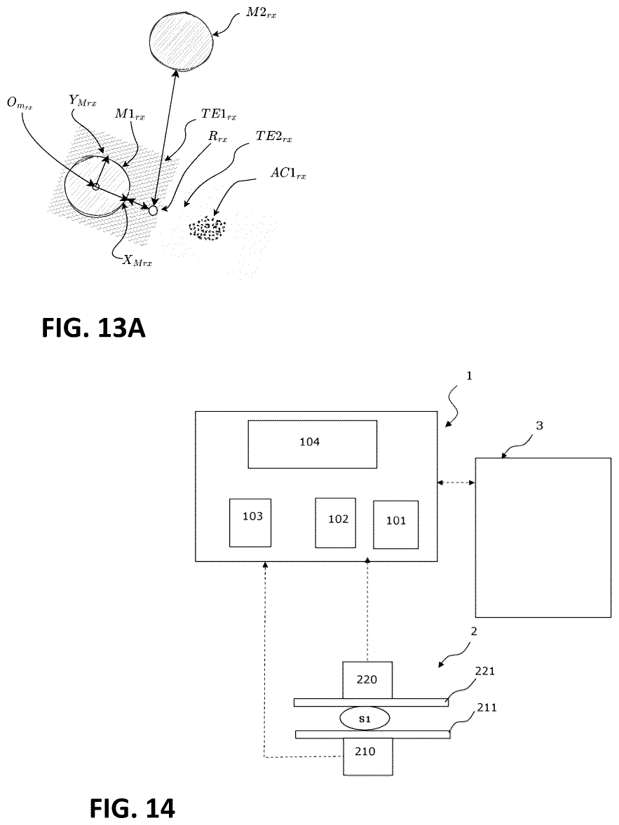

Preferably, as illustrated in the example of FIGS. 12 and 13 and 13A, the correlation function is also a function of the type of tissue TE1rx, TE2rx (fatty or fibro-glandular) containing the point of the pointing device of the source image whose corresponding position is to be determined in the destination image. The tissues TE1rx, TE2rx in the source image correspond to the tissues TE1irm, TE2irm in the destination image. Said tissue also can be of the muscle tissue or calcified tissue type.

The point Rirm that in the image IMirm corresponds to the point Rrx in the image IMrx is obtained by applying the correlation function to the point Rrx.

In the coordinate system associated with the internal structure Mirm, the obtained coordinates for Rirm are R_Mirm_x=10 mm along the axis XMirm, and R_M_irm_y =5 mm along the axis YMirm.

The system also allows the coordinates of Rirm to be obtained in the associated coordinate system within the image IM_irm, which, in the example, are the coordinates R_S1_irm_x=20 mm along the axis PM, and R_S1_irm_y=30 mm along the axis PC.

In the case, which does not form part of the invention, whereby determining the position of the point Rrm in the image IM_irm that corresponds to the point Rrx in the image IM_rx would be carried out not with the correlation function considering the pair of reference internal structures, but by applying a general deformation model of the breast to the breast S1 of the image IMrx in order to attain the breast of the image IMirm, it has been observed that determining the coordinates of the point Rirm′ that would then be obtained would be erroneous relative to the coordinates of the point Rirm obtained with the correlation function considering the pair of reference internal structures. By way of an example, the obtained point Rirm′ would have the following coordinates R_S1_irm′_x=30 mm along the axis PM, and R′_S_irm′_y =30 mm along the axis PC (reference frame of the breast S1).

When the source is acquired with a lower resolution than the destination acquisition, considering a pair of reference internal structures allows the level of accuracy to be increased for positioning the locating pointer on the acquired destination image. FIG. 11 illustrates the precise positioning of the pointer on the image IMrx of the destination at a point Rrx, corresponding, in the source image IMirm, to the positioning point Rim of the pointer of the pointing device (such as a mouse), with the point Rrx being obtained by considering the internal structures StInt1irm; StInt1rx. In particular, the coordinates of the source point in the reference frame Omirm, Xmirm, Ymirm are considered, with said reference frame being associated with the internal structure StInt1irm. Thus, in the example illustrated in FIG. 11, precise positioning of the pointer is obtained in the acquired destination image.

When multiple internal structures are identified in the source image of the breast that match the internal structures of the destination image (i.e., several pairs of internal structures are detected), the type of internal structure is considered for selecting the reference internal structure associated with a reference frame. Provision can be made for some types of determined internal structures to be prioritized over other types of determined internal structures when selecting the reference internal structure.

According to one embodiment of the invention, in terms of selection as a reference internal structure, a mass-like internal structure is prioritized over a calcification cluster-like internal structure. Indeed, a mass-like internal structure represents a more stable reference frame anchor in the breast than a calcification cluster. Preferably, a calcification cluster-like structure is prioritized over a set of fibers with architectural disorganization. Advantageously, a set of fibers with architectural disorganization is prioritized over a vessel-like structure.

According to one embodiment of the invention, fatty tissue is excluded from candidates for selecting a reference internal structure.

In the event that several pairs of internal structures of the same type are identified, in particular several masses, the distance between the pointer and each internal structure (for example, each mass) is considered so as to select the internal structure that is closest to the point. In particular, it is possible to consider the distance between the point and an edge of the internal structure in order to determine said distance. According to a particular aspect, the pointer is located in the source image of the breast outside the internal structures.

Thus, when the pointer is positioned between multiple identified internal structures, considering the type of structure, and optionally the distance between the point and the identified internal structures of the same type, allows the pointer to be more precisely positioned on the destination image. An example of such a case is illustrated in FIG. 13 (with a detailed view of the internal structures being shown in FIG. 13A, which also schematically shows the possible presence of an additional mass), with a source being acquired using X-ray in which mass-like and calcification cluster-like internal structures have been identified. In this example, a reference frame associated with the mass M1rx is selected rather than the calcification cluster AC1rx because the mass is considered to be more stable and less subject to deformation than the calcification cluster, which allows a position Rirm to be obtained for the pointer in the image IMirm that corresponds to the point Rrx for positioning the pointer in the image IMrx, which is more reliable.

Assuming that multiple internal structures of the same type would be present, for example, masses, as is schematically illustrated in FIG. 13A with the added representation of a mass M2rx in this figure, the selection between the two structures (masses) is made as a function of the distance so that the mass that is selected as the reference structure is the mass M2rx that is closer to the point Rrx than the mass M2rx.

As previously noted, in order to establish the correlation function for determining the position of the point Rirm in the destination image that corresponds to the point Rrx in the source image, it is also possible to consider the type of tissue TE1rx in which the point Rrx (and the point Rirm in the destination image) is located. Indeed, the tissue in which the point Rrx is located can be of the fatty or fibrous type and a fatty tissue is more deformable than a fibrous tissue, such that considering the type of tissue (in particular the associated physical property of density and/or elasticity) and a deformation parameter (gravitational force or force applied to the breast by an appliance) experienced by the breast in the configuration for acquiring the destination image, and/or a deformation parameter experienced by the breast in the configuration for acquiring the source image (considering the two deformation parameters allows a relative deformation to be considered), allows the position of the point Rirm in the destination image to be determined more reliably.

When several internal structures are overlaid on the source acquisition, provision can be made for the position of the pointer on the overlaid structure to be analyzed in order to determine the structure most likely to be targeted by the pointer, in other words, the structure that the operator is likely to point toward (or select) by positioning their pointer on the overlaid structure. Determining the targeted structure and therefore the structure to be selected, then can be carried out as a function of the type of structure and the position of the pointer relative to the structure.

### Synchronization of Pointers

As noted above, the pointer that can be moved in the source image is, for example, a cursor of a pointing device, such as a mouse, a trackpad, or a stylus.

When the pointer of the pointing device points to a point in the source image, the processing unit implements the steps described hereafter in order to identify, on the destination image, the corresponding point using the correlation function described above. As previously explained, once the reference internal structure is defined or selected, the correlation function determines said position of the point in the reference frame attached to the reference internal structure of the destination image, at least as a function of the position of the point of the pointing device in the reference frame of the reference internal structure of the source image, of the distance of the point to be determined relative to the external structure in the destination image (particularly to force the searched point not to be located in the destination image outside the breast), and preferably as a function of the type of tissue in which the point is located. The processing unit then displays the position of the pointer associated with the destination image on this point. In other words, pointing the point of the source image causes a pointer, such as a cursor, to be positioned on the corresponding point of the destination image.

The method and the corresponding system thus allow each point of the breast on an acquired image to reliably and precisely find the match for this point for each of the other acquired images of this breast. It is thus possible to establish the link between the acquisitions in order to allow inter-acquisition spatial browsing that is reliable and precise.

### Processing Unit

The steps of analyzing or processing images and of synchronizing the locating pointers on the images are carried out by a processing unit.

The processing unit is, for example, in the form of a processor and a data memory storing computer instructions that can be executed by said processor, or even can be in the form of a microcontroller.

In other words, the described functions and steps can be implemented in the form of a computer program or via hardware components (for example, programmable gate arrays). In particular, the functions and steps performed by the processing unit can be carried out by sets of computer instructions or modules implemented in a processor or controller or can be carried out by dedicated electronic components or FPGA or ASIC type components. It is also possible to combine computer parts and electronic parts.

Thus, the processing unit is an electronic and/or computer unit. When said unit is specified as being configured to carry out a given operation, this means that the unit comprises computer instructions and the corresponding execution means for carrying out said operation and/or that the unit comprises corresponding electronic components.

According to one embodiment and as described above, the processing unit allows all or some of the various processing operations defined below to be carried out:
filtering, which is a processing operation applied to an image that is intended to modify the values assigned to each point of the image in a reversible or irreversible manner, in order to improve the interpretability of the image for the subsequent processing operations. The filtering can include, for example, noise attenuation or contrast enhancement;
segmenting, which is a processing operation applied to an image that is intended to define one or more groups of points (for example, regions, or segments) meeting similar characteristics;
classifying, which is a processing operation applied to an image intended to assign the classes or type (for example, numerical or logical values), to an entire image or to an image portion;
correlating, which is a processing operation applied to points of multiple images in order to establish spatial relationships between the points of the various images;
spatial locating, which is a processing operation intended to view the correlation results concerning the set of acquisitions.

The spatial locating allows, when an operator selects or points to an area (or a point) on an acquired image, the one or more corresponding areas (or points) to be determined in the one or more other acquired images and allows a pointer to be displayed, such as a corresponding cursor, on said one or more corresponding areas (or points).

The invention is not limited to the embodiments illustrated in the drawings. Accordingly, it should be understood that, when the features mentioned in the appended claims are followed by reference signs, these signs are only included to improve the intelligibility of the claims and by no means limit the scope of the claims.

In addition, the term “comprising” does not exclude other elements or steps. Furthermore, features or steps that have been described with reference to one of the embodiments disclosed above also can be used in combination with other features or steps of other embodiments disclosed above.

1. A computer-assisted method for synchronously positioning a locating pointer (Rrx, Rirm) in various acquired images (IMrx, IMirm) of the same deformable breast, the method comprising the following steps of:
acquiring a first image, called source image, of the breast according to a first image acquisition configuration;
acquiring a second image, called destination image, of the breast according to a second image acquisition configuration, distinct from the first configuration;
detecting at least one external structure of the breast in the source image and at least one matching external structure, such as the thoracic cage and the skin of the breast, of the breast in the destination image;
detecting and classifying an internal structure of the breast, such as a mass inside the breast, in the source image;
identifying an internal structure in the destination image that corresponds to the internal structure detected in the source image,
with said internal structures forming a pair of internal structures;
defining a reference frame attached to said internal structure for each image, with said internal structure to which the reference frame is attached being called reference internal structure;
defining, for several points of the source image, a correlation function for matching one of said points of the source image with a point of the destination image, with the correlation function being established at least as a function of the coordinates of said point of the source image in the reference frame attached to the reference internal structure of the source image, and as a function of the reference frame attached to the reference internal structure of the destination image;
with said correlation function also being established as a function of the distance of said point of the destination image relative to said at least one external structure of the destination image;
displaying, using a graphical user interface, the source image and the destination image;
determining, for a pointer of a pointing device, such as a mouse, positioned by an operator using the graphical user interface on a point of the source image, the corresponding point of the destination image (IMirm) using said correlation function and displaying a pointer on said corresponding point of the destination image.

2. The method as claimed in claim 1, wherein, with several pairs of internal structures being identified, the method comprises, for establishing said correlation function, selecting one of the pairs of internal structures, as a pair of reference internal structures, as a function of the type of internal structure of each pair, such as the mass; and, when the pairs of internal structures include several pairs of internal structures of the same type, the pair of reference internal structures is selected by considering, in the source image , the distance of the point relative to the internal structure of each pair so as to select the internal structure whose distance between said internal structure and said point is the shortest.

3. The method as claimed in claim 1, wherein the first image acquisition configuration and/or the second image acquisition configuration includes a breast deformation parameter, such as a breast compression force applied by an appliance or the gravitational force experienced by the breast, such that the breast in the destination image assumes a shape different from the shape assumed by the breast in the source image;
with said correlation function also being established as a function of said one or more breast deformation parameters.

4. The method as claimed in claim 1, wherein the method comprises determining the type of tissue in which said point of the source image is located,
with said correlation function also being established as a function of at least one physical property of said determined tissue type.

5. The method as claimed in claim 4, wherein said tissue is fatty tissue, fibro-glandular tissue, muscle tissue or calcified tissue.

6. The method as claimed in claim 4, wherein said at least one physical property of said type of tissue in which the point is located and which is used to establish the correlation function is the density or the elasticity.

7. The method as claimed in claim 1, wherein a mass-like internal structure has a density within a range of predefined values, for example, [1.01; 1.08] g/ml.

8. The method as claimed in any claim 1, wherein said external structure used to establish the correlation function comprises the skin of the breast that defines the contour of the breast.

9. The method as claimed in claim 1, wherein the first image acquisition configuration and the second image acquisition configuration differ from each other by at least one of the following parameters:
the acquisition technology used;
the acquisition parameters or conditions used;
the acquisition time.

10. The method as claimed in claim 1, wherein the first image acquisition configuration and the second image acquisition configuration comprise at least one of the following acquisition methods:
X-ray imaging;
magnetic resonance imaging;
ultrasound imaging.

11. The method as claimed in claim 1, wherein the first image acquisition configuration and the second image acquisition configuration comprise, on the one hand, an X-ray imaging or magnetic resonance imaging acquisition method, and, on the other hand, an ultrasound imaging acquisition method.

12. The method as claimed in claim 1, wherein the internal structures to be classified comprise:
one or more anatomical structures, such as a sub-mammary furrow;
one or more physiological structures, such as a vessel; and/or
one or more pathological structures, such as a mass, micro-calcifications or macro-calcifications.

13. A non-transitory computer program product comprising program code instructions for executing the steps of a method as claimed in claim 1, when said program is executed by a processor of a computer system.

14. A computer system comprising a memory containing program code instructions for executing the steps of a method as claimed in claim 1, and a processing unit comprising a processor for executing said program code instructions, said computer system further comprising:
a memory for storing image data;
a pointing device, such as a mouse, that can be handled by an operator,
a display screen; and
a graphical user interface configured to:
simultaneously and separately display a first acquired image, called source image, and a second acquired image, called destination image, of a breast;
display, on a point of the source image, a pointer as a function of the position of the pointing device handled by the operator;
display, following the execution of said program code instructions, another pointer on the acquired destination image at a point on said destination image that corresponds to the point of the source image on which the pointer of the pointing device handled by the operator is positioned.

15. A facility comprising:
a computer system as claimed in claim 14;
a first imaging appliance for acquiring the source image; and
a second imaging appliance, different from the first imaging appliance, for acquiring the destination image.

16. The facility as claimed in claim 15, wherein said first imaging appliance comprises an X-ray appliance that comprises an emitter device (210) able to emit an X-ray beam and a receiver device able to receive the X-rays emitted by the emitter device after they pass through the breast, with the X-ray appliance being configured to transmit image data to the computer system that corresponds to the X-rays received by the receiver device after they pass through the breast.

17. The facility as claimed in claim 15, wherein said second imaging appliance comprises a magnetic resonance appliance that comprises an emitter device able to emit magnetic radiation and a receiver device able to receive the magnetic radiation emitted by the emitter device after it passes through the breast, with the magnetic resonance appliance being configured to transmit image data to the computer system that corresponds to the magnetic radiation received by the receiver device after it passes through the breast.

18. The method as claimed in claim 1, wherein the pair of reference internal structures is selected by considering, an edge of the internal structure, of each pair so as to select the internal structure whose distance between said internal structure and said point is the shortest.

19. The method as claimed in claim 18, wherein the pair of reference internal structures is selected by considering, an edge of the internal structure, of each pair so as to select the internal structure whose distance between said edge of said internal structure and said point is the shortest.

20. The method as claimed in claim 7, wherein a mass-like internal structure has a density within a range of [1.01; 1.08] g/ml.
